(19) United States
(12) Patent Application Publication
(10) Pub. No.: US 2023/0105668 A1
(43) Pub. Date: Apr. 6, 2023

(54) MICROPROCESSING FOR PREPARING MODIFIED PROTEIN

(71) Applicant: CARGILL, INCORPORATED, Wayzata, MN (US)

(72) Inventors: Fabienne BOSSARD, Vilvoorde (BE); Ralf JAKOBI, Vilvoorde (BE); Christof KUESTERS, Vilvoorde (BE); Laurice POUVREAU, Bennekom (NL)

(73) Assignee: CARGILL, INCORPORATED, Wayzata, MN (US)

(21) Appl. No.: 17/907,127

(22) PCT Filed: Mar. 26, 2021
(86) PCT No.: PCT/US2021/024427

§ 371 Date: Sep. 23, 2022

(30) Foreign Application Priority Data
Mar. 26, 2020 (EP) 20165816.8

(51) Int. Cl. C07K 1/107; C07K 14/415; A23L 33/185; A23K 20/147; A23J 3/18

(52) U.S. Cl. CPC C07K 1/1077; C07K 14/415; A23L 33/185; A23K 20/147; A23J 3/18; A23V 2002/00

(57) ABSTRACT

The invention relates to the use of a microdevice for the modification of protein with carbohydrate. Preferably for the glycation of protein with a mono-, di-, oligo- or polysaccharide(s). The invention also relates to the process for modifying protein with carbohydrate in a microdevice. The invention also relates to a process for preparing a food, feed, personal care, cosmetic, pharmaceutical, paper or corrugated board product comprising the process steps to prepare the modified protein and the step of combining the modified protein with at least one other ingredient.

### CROSS-REFERENCE TO RELATED APPLICATIONS

This application claims the benefit of European Patent Application No. 20165816.8, filed Mar. 26, 2020, which is hereby incorporated by reference in its entirety.

### FIELD OF THE INVENTION

The present invention relates to modified proteins, in particular glycated proteins obtained from a process using microdevices. The invention relates in particular to a method of obtaining such glycated proteins in a microdevice/microreactor.

### BACKGROUND OF THE INVENTION

Proteins occupy a unique position in the world of biological matter due to their relatively large size and complex structures. Proteins are an important ingredient in the food & feed industries due to their abundant nutritive values, particularly as a provision of essential amino acids that are not synthesized in the human body. Proteins under specific conditions can act as emulsifiers acting strongly at the oil-water interface. They are already used to a certain extent in the stabilization of oil-in-water emulsions.

However, proteins are unstable under certain conditions. The functional properties of many proteins allowing their use as emulsifiers is easily lost under acidic conditions, high ionic strength, high temperature, and/or in the presence of organic solvents. This limits their industrial applications. Especially at low pH, proteins tend to precipitate due to decreased solubility in the solution. When used in beverages, this precipitate may also contribute to a perceived stronger astringent taste, and furthermore sedimentation and suspensions are often regarded by the consumer as unappealing.

Thus, proteins need to be converted into more stable forms in order to have a more versatile use in the food industry and beyond, for example in new functional formulations to incorporate bioactive compounds into food or feed matrices and even into personal care products. Functional properties of proteins can be improved through physical, chemical or enzymatic treatments.

An emulsifier must have amphiphilic properties (possessing both hydrophobic and hydrophilic groups) to reduce the surface tension between two liquids. One way of strengthening this property in a protein is by glycation, a type of Maillard reaction, whereby the reducing end of a sugar and an amino acid of a protein become covalently linked to form a glyco-conjugate or glycated protein.

High molecular weight glyco-conjugates possess the properties of the protein, strongly adsorbing at the surface of oil droplets and also possess the hydrophilic properties of the sugar, allowing solvation in an aqueous medium. The conjugation between proteins and sugars provides much more improved steric stabilization of the emulsion droplets over a wide range of environmental conditions, such as low pH and high ionic strength.

Glycation can thus enhance not only the emulsifying properties of proteins, but also their solubility, thermal stability and foaming capacity. For instance, the oxidative stability of fish oil microcapsules has been reported to be largely improved by glycated soybean protein isolate (Zhang et al., Food Hydrocolloids, 51, 108-117, 2015). Casein-carrageenan conjugates were also reported to increase the thermal, intestinal and storage stability of microcapsules for encapsulation of the red pigment from paprika (Qiu et al., Carbohydrate Polymers, 196, 322-331, 2018). Evidence thus shows that glycation could be an efficient and safe strategy for enhancing the different functionalities of proteins.

However, glycated proteins are very difficult to prepare. Maillard reactions are known to proceed at a higher rate in the dry state than in the liquid/solvated state (Schroeder, Iacobellis, & Smith, 1955). According to the conventional approach, glycation is thus done in the dry or semidry state (e.g. WO2011/059330). However, still the reaction takes several days to complete. For instance, Wang et al. discloses freeze-dried glycation, whereby a protein and sugar are mixed together in water and then centrifuged to obtain a supernatant, which is then freeze-dried. The resulting freeze-dried mixture is then incubated at 60° C. for 3 days at a relative humidity of 79% to carry out glycation (Wang et al., Food Research International, 119 (2019) 227-235).

Modification of proteins by wet heating in solutions have also been reported (e.g. WO2009/117572 or Zhu et al., Journal of Agricultural and Food Chemistry 2010). Xu et al. discloses reacting myofibrillar protein (1.00 w/v %) and dextran (1.00 w/v %) at three different molecular weights individually mixed in 20 mmol/L phosphate-buffered saline solution (pH 7.5). The reaction was allowed to continue at 37° C. for 8 h under constant agitation. However, reactions between the dextran and protein were limited. Table 1 describes the degree of protein modification varying from only 4.3 to 8.8% (Xu et al. LWT—Food Science and Technology 117 (2020) 108664). The Maillard reaction still occurs slowly in aqueous solution, possibly due to the retarded formation of water molecules during Amadori rearrangement (M. H. Abd El-Salam, S. El-Shibiny/International Journal of Biological Macromolecules 112 (2018) 83-92). Neither the dry nor the wet state glycation reactions are economically feasible on an industrial scale.

Several new technologies, such as pulsed electric field processing (Sun et al. Food Research International, 44(4), 1052-1058, 2011), subcritical water processing (Plaza et al., Food Research International, 43(4), 1123-1129, 2010), ultrasound treatment (Liu et al., LWT-Food Science and Technology, 109, 130-136, 2019 or Corzo-Martinez et al., Journal of dairy science 2011), high pressure microfluidization (Ozturk et al., Innovative Food Science & Emerging Technologies, Volume 52, p. 179-188, 2019) and microwave irradiation (MI) (Nasrollahzadeh et al., Food Research International, 100, 289-297, 2017) have been applied for glycation reaction recently. However, none of these overcome the problem of slow and/or incomplete reactions.

Other glycation reactions are disclosed in WO2013067603 and WO2009117572.

Such procedures thus may take many hours, even days and may require energy intensive steps, such as freeze-drying, which is not suitable on an industrial scale.

There is thus a need to develop a more efficient means to prepare glycated proteins. The new means should provide one or more of: a higher yield, shorter processing time, reduced formation of by-products and more consistent product quality.

There is a tendency towards manufacturing smaller-scale equipment due to the desire for size efficiency. Recently, scientists have learned that not only electronic devices, but also mechanical devices, may be miniaturized and batch-fabricated, promising the same benefits to the mechanical world, as integrated circuit technology has given to the electronic world. These are called microreactors. In the past decade, microreactor technology, as a merging of microfluidic chemistry and continuous-flow technology, has begun to be explored (see for example, Fletcher et al., Tetrahedron, Vol. 58, no. 24, June 2002 or EP2433970A1 for the preparation of polycondensates). No microreactors are being used yet on an industrial scale in the food industry.

Microreactors have been used in small-scale lab synthesis of oligosaccharides i.e. glycosylation of saccharides to form longer carbohydrate chains (Seeberger et al.; Organic Letters 2007, Vol. 9, No. 12, 2285-2288). Glycosylation in a microreactor is described by Seeberger et al. in various publications. However, these publications are about linking carbohydrates together to synthesize oligosaccharide chains. This “glycosylation” is not the same as protein glycation i.e. covalently linking sugars to a protein (for instance to a lysine amino acid residue in the protein). The prior art, in particular Seeberger et al., does not teach the use of microreactors for carrying out reactions of carbohydrates with proteins. To date, microreactors have not been used for proteins in order to modify their properties.

D'Ulivo et al. (Analytica Chimica Acta 664 (2010) 185-189 discloses an open tubular capillary electrochromatography, whereby the capillaries are coated with collagen and then filled with a glucose phosphate solution. The reaction was carried out for 8, 16 or 24 hours at 37° C. The glycated collagen remained as a coating in the capillary for further analytical purposes. Although referred to as a microreactor due to the size of the equipment, this is not a microreactor suitable for producing glycated proteins as an end product. The system does not comprise any micro heat-exchangers or micro-mixers and is not suitable for rapid glycation reactions at elevated temperatures (>60° C.) and short residence times (less than 150 seconds). The disclosure in D'Ulivo et al. is broadly similar in concept to the capillary disclosed in US2006/199945, which discloses using dextran coated capillaries to synthesize short chain amino acids; again this is not a system suitable for the efficient production of glycated proteins as an end product and does not teach the skilled person that microdevices are an efficient means to glycate proteins.

One of the main reasons is that the skilled person would not expect that large protein molecules suspended in aqueous media can be transferred through the capillaries of a microreactor, without creating blockages and fouling. Herein the applicant will demonstrate that surprisingly the opposite was in fact observed, thus providing a convenient and efficient solution for the problem of glycating proteins in a manner that can be industrially applicable on a large scale.

### SUMMARY OF THE INVENTION

The current invention relates to a process for preparing modified protein comprising the following steps:
a) Mixing a composition of protein(s) (A) and a composition of carbohydrate(s) (B) to form a composition (C) in an aqueous medium;
b) Optionally, adjusting the pH of the composition (C), preferably to a pH of from 6 to 9, more preferably from 6.5 to 9, even more preferably from 7 to 8.5, most preferably from 7 to 8;
c) Adding the composition (C) into a microdevice;
d) Reacting the protein(s) with the carbohydrate(s) in the microdevice to obtain a composition of modified protein(s) (D).

The composition of modified protein(s) (D) can be recovered.

Optionally, the composition of protein(s) (A) is a composition comprising one or more of plant-based protein(s), dairy protein(s), single cell protein(s), and fungal protein(s). Preferably, the composition of protein(s) (A) is a composition comprising one or more of dairy, cereal and legume protein(s). More preferably, the composition of protein(s) (A) is a composition comprising one or more of whey, wheat, corn, soybean and pea protein(s), most preferably one or more of whey, wheat and soybean protein(s). Most preferably the composition of protein(s) (A) is a composition comprising soluble wheat protein(s) or soluble hydrolyzed wheat protein(s).

Optionally, the composition of carbohydrate(s) (B) is a composition comprising, essentially consisting of or consisting of one or more of monosaccharide(s), disaccharide(s), and oligosaccharide(s).

Optionally, the composition of carbohydrate(s) (B) is a composition comprising, essentially consisting of or consisting of one or more of glucose, allulose, mannose, fructose, rhamnose, galactose, maltose, lactose, lactulose, and isomaltose. Preferably the composition of carbohydrate(s) (B) comprises, essentially consists of or consists of glucose.

Optionally, the microdevice comprises micro-heat exchanger(s) and/or micro-reactor(s), and optionally micro-mixer(s), and is suitable for the reaction of protein with carbohydrate.

Optionally, the weight ratio of composition of protein(s) (A) to composition of carbohydrate(s) (B) in the composition (C) ranges from 1:10 to 10:1, preferably 1:5 to 5:1, more preferably from 1:1 to 5:1, most preferably around 1:1.

Optionally, the composition (C) has a dry substance content of from 5 to 50 wt %, preferably 10 to 40 wt %, even more preferably 20 to 35 wt %, most preferably around 30 wt %.

Optionally, the pH is adjusted in step (b) with a base, preferably sodium hydroxide.

Optionally, the reaction in step (d) takes place in the microdevice:
at a temperature of from 60 to 120° C., preferably 70 to 110° C., more preferably 80 to 100° C., most preferably 85 to 95° C. and
for a duration of less than 150 seconds, preferably less than 120 seconds, more preferably less than 110 seconds, even more preferably less than 100 seconds, most preferably from 1 to 90, 80, 70, 60, 50, 40, 30, 20 or 10 seconds.

Optionally, the method further comprises step (e) wherein the composition of modified protein(s) (D), optionally mixed with a further composition of carbohydrate(s) (B′), which can be the same or different from the composition (B), is reinjected back into the same microdevice and/or into a second microdevice to increase the degree of modification of protein(s) in order to obtain a composition of modified protein(s) (D′).

Optionally, the method further comprises step (f) wherein the composition of modified protein(s) (D) or (D′) is:
optionally purified and
incorporated into a food, feed, personal care, cosmetic, pharmaceutical, paper or corrugated board product comprising at least one other ingredient.

The invention also covers a glycated protein, namely wheat or soybean protein glycated with carbohydrate(s), preferably mono-, di-, oligo- or polysaccharide(s), preferably with one or more of glucose, mannose, galactose, rhamnose, fructose, maltose, isomaltose, maltulose, mannobiose and lactose, more preferably glycated with glucose.

The invention also covers a composition of modified protein(s) (D) or (D′) obtainable according to the method of the invention.

The invention also covers a food, feed, personal care, cosmetic, pharmaceutical, paper or corrugated board product comprising the optionally purified composition of modified protein(s) (D) or (D′) obtainable according to the method of the invention and at least one other ingredient. Preferably, the composition of modified protein(s) (D) or (D′) comprises wheat or soybean protein glycated with mono-, di-, oligo- or polysaccharide(s), preferably with glucose.

The invention also covers a food, feed, personal care, cosmetic, pharmaceutical, paper or corrugated board product comprising glycated wheat or soybean protein according to the invention and at least one other ingredient.

The invention also relates to a process for preparing a food, feed, personal care, cosmetic, pharmaceutical, paper or corrugated board product comprising the process for preparing a composition comprising modified protein(s) (D) as stated above and the step of combining the composition comprising modified protein(s) (D) with at least one other ingredient.

The current invention further relates to the use of a microdevice for the modification of protein(s) with carbohydrate(s).

The current invention further relates to the use of a microdevice for the glycation of protein with a mono-, di-, oligo- or polysaccharide(s), preferably wherein the microdevice comprises micro-heat exchangers and/or micro-reactors and optionally micro-mixer(s), and is suitable for the reaction of protein with carbohydrate, preferably with a reaction duration of less than 150 seconds, and preferably at a reaction temperature of at least 60° C.

### BRIEF DESCRIPTION OF THE DRAWINGS

FIGS. 1 and 2 detail the structure of an example microreactor system used in the Examples 1 and 2.

FIGS. 3 to 6 represent different reaction conditions and results obtained from carrying out the Examples 1 and 2 as described below.

FIG. 7 shows the process flow diagram of a lab-scale 8 channel microreactor system.

FIG. 8 illustrates the geometric configuration of a microreactor. FIGS. 7 and 8 are originally disclosed by Sadir et al. in the publication “Numerical and Experimental Investigation of Flow Maldistribution due to Blockage in Micro structured Heat Exchanger” in the Journal of Fluid Flow, Heat and Mass Transfer (JFFHMT), Volume 8, 2021 (publication in progress).

### DETAILED DESCRIPTION OF THE INVENTION

The current invention preferably relates to a process for preparing modified protein comprising the following steps:
a) Mixing a composition of protein(s) (A) and a composition of carbohydrate(s) (B) to form a composition (C) in an aqueous medium;
b) Optionally adjusting the pH of the composition (C), preferably to a pH of from 6 to 9, more preferably from 6.5 to 9, even more preferably from 7 to 8.5, most preferably 7 to 8;
c) Adding the composition (C) into a microdevice;
d) Reacting the protein(s) with the carbohydrate(s) in the microdevice to obtain a composition of modified protein(s) (D).

The composition of modified protein(s) (D) can be recovered.

The composition of modified protein(s) (D) can be recovered and optionally purified before being used in food, feed, personal care, cosmetic, pharmaceutical, paper or corrugated board products with at least one other ingredient.

Preferably, the microdevice comprises micro-heat exchanger(s) and/or micro-reactor(s), and optionally micro-mixer(s), suitable for the reaction of protein with carbohydrate.

### 1. The Composition of Protein(s) (A)

The composition of protein(s) (A) preferably comprises from 50 to 99.9 wt % of proteins on a dry weight basis, more preferably from 60 to 99 wt %, even more preferably from 65 to 98 wt %, most preferably from 70 to 98 wt %. The protein content is measured on the basis of total nitrogen content multiplied by the factor 6.25. Nitrogen content can be measured using the Dumas method, for example using a LECO® CN928 analyzer.

The composition of protein(s) (A) may comprise one or two or more different types of proteins.

The compositions of protein(s) (A) may comprise soluble proteins.

The compositions of protein(s) (A) may comprise partially soluble or non-soluble proteins. The particle size of partially soluble or non-soluble proteins should be substantially smaller than the channel diameter of the microreactor to avoid blockage. The skilled person knows what size the particles need to be depending on the size of the channels.

The composition of protein(s) (A) may comprise protein from any known source. Preferably the composition of protein(s) (A) comprises protein from plant-based sources, animal sources, single-cell protein sources, fungal sources and any combination of two or more thereof. The amounts of protein in the composition (A) as disclosed above apply to any of the compositions of protein(s) (A) according to the invention.

### 1.1. Plant-Based Sources

Plant-based sources of protein include cereals (including pseudocereals), legumes (i.e. pulses), nuts, seeds and vegetables.

Examples of cereals and pseudocereals, as sources of proteins, include wheat, buckwheat, oats, rye, millet, maize (corn), rice, sorghum, amaranth, quinoa etc.

Examples of legumes, nuts and seeds, as sources of proteins, include soybeans, lentils, kidney beans, white beans, fava beans, mung beans, chickpeas, green peas, cowpeas, lima beans, edamame, pigeon peas, lupines, wing beans, pinto beans, almonds, Brazil nuts, cashews, pecans, pistachios, walnuts, cotton seeds, rapeseed, pumpkin seeds, hemp seeds, chia seeds, sesame seeds, sunflower seeds, flax seeds, camelina seeds etc.

Examples of vegetables, as sources of proteins, include roots and tubers such as potatoes, yams, cassava, sweet potatoes and other vegetables such as brussels sprouts, yellow sweet corn, asparagus, broccoli, avocado etc.

Preferably, the composition of protein(s) (A) comprises wheat protein. Without being bound by theory, it is known that wheat proteins typically comprise albumin, globulin, gliadin and glutenin.

Preferably, the composition of protein(s) (A) comprises soybean protein. The composition of protein(s) includes soybean protein in the form of soybean meal, soybean flour, soybean protein isolate, soybean protein concentrate etc.

### 1.2. Animal Sources

Animal sources of protein include animal meat including seafood and fish (e.g. muscle tissue, connective tissue), animal blood plasma, dairy, and eggs.

Examples of proteins from dairy sources include whey proteins and casein.

When whey proteins are isolated from whey protein, they are referred to as whey protein isolate (WPI). An example thereof is Bi-Pro® WPI.

Preferably, the composition of protein(s) (A) comprises whey protein. Without being bound by theory, it is known that whey proteins are a mixture of globular proteins isolated from whey containing beta-lactoglobulin, alpha-lactalbumin and serum albumin

### 1.3. Hydrolysis and Solubility

Any of the above-mentioned protein compositions can also be (partially) hydrolyzed protein composition. By hydrolyzed proteins, it is meant herein a degree of hydrolysis of from 1 to 75%, preferably of from 1 to 50%, more preferably of from 1 to 30%, even more preferably of from 1 to 25%, yet more preferably 1 to 20%, and most preferably 1 to 15%. The degree of hydrolysis can be determined using the OPA method (“Improved method for determining food protein degree of hydrolysis”, P. M. Nielsen, D. Petersen and C. Dambmann J of Food Sciences, 2001, Vol. 66, no 5, p. 642-6465).

An example of a hydrolyzed protein is hydrolyzed wheat protein. Thus, the composition of proteins (A) may be a composition comprising hydrolyzed wheat protein. The degree of hydrolysis can be of from 1 to 75%, preferably of from 1 to 50%, more preferably of from 1 to 30%, even more preferably of from 1 to 25%, yet more preferably 1 to 20%, and most preferably 1 to 15%.

Any of the protein compositions according to the invention may also be a composition of solubilized or soluble (hydrolyzed) proteins. By a composition of solubilized or soluble proteins, it is meant herein that the protein composition was treated in order to remove at least partially any insoluble fraction(s) from the composition.

An example of a solubilized and hydrolyzed protein is solubilized hydrolyzed wheat protein. Thus, the composition of proteins (A) may be a composition comprising solubilized hydrolyzed wheat protein. An example of solubilized/soluble hydrolyzed wheat protein compositions is provided in EP2117338B1, which is incorporated herein by reference. The degree of hydrolysis can be of from 1 to 75%, preferably of from 1 to 50%, more preferably of from 1 to 30%, even more preferably of from 1 to 25%, yet more preferably 1 to 20%, and most preferably 1 to 15%.

However, the invention is by no means limited to compositions of soluble or hydrolyzed proteins only.

### 2. The Carbohydrate(s)

The composition of carbohydrate(s) (B) preferably comprises from 10 to 99.9 wt %, or 15 to 99 wt %, or 20 to 90 wt %, or 20 to 80 wt % of carbohydrate(s) on a dry weight basis.

The carbohydrate(s) can be any carbohydrate as long as the carbohydrate(s) has at least one reducing end. By “reducing end” it is meant herein a free aldehyde or ketone functional group allowing the carbohydrate to react as a reducing agent. Preferably, the carbohydrate(s) can be any monosaccharide, disaccharide, oligosaccharide, polysaccharide and any combination of two or more thereof, as long as the carbohydrate(s) has at least one reducing end. The reducing end is needed for the sugar to react with the protein.

The composition of carbohydrate(s) (B) can be in solid form or liquid form e.g. in the form of a syrup.

Preferably the composition of carbohydrate(s) (B) comprises, essentially consists of or consists of carbohydrate(s) that is selected from one or more of a monosaccharide, a disaccharide, an oligosaccharide or a polysaccharide. More preferably the carbohydrate(s) is selected from one or more of a monosaccharide and/or a disaccharide i.e. preferably a saccharide with a degree of polymerization of less than 3. The larger the carbohydrate, the more limited the reactivity between the protein and the carbohydrate and the lower the efficiency of glycation.

Examples of monosaccharide(s) include rhamnose, glucose, mannose, fructose, galactose, arabinose, allulose, allose, altrose, gulose, iodose, talose, deoxyribose, ribose, xylose, lyxose and combinations of two or more thereof. More preferably the monosaccharide(s) is(are) selected from glucose, allulose, mannose, fructose, rhamnose and galactose. More preferably the monosaccharide comprises glucose. Most preferably the monosaccharide consists essentially of or consists of glucose.

Glucose can be provided in solid form or liquid form, whereby the solid form is either a solidified form or a crystalline form. Further suitable sources of glucose are glucose syrups comprising from 50 wt % to 99.9 wt %, preferably from 60 wt % up to 99 wt % of glucose, more preferably from 70 wt % up to 90 wt % of glucose, on a dry weight basis. The remaining components in the glucose syrup are residual oligomers such as maltose, maltotriose and higher glucose polymers.

Preferably, the composition (B) essentially consists of or consists of glucose. Glucose is also known in the art as dextrose.

Most preferably, the composition of carbohydrate(s) (B) is in solid form comprising, essentially consisting of or consisting of crystalline glucose, preferably a glucose monohydrate.

Examples of disaccharide(s) include maltose, lactose, rutinose, gentiobiose, cellobiose, isomaltose, lactulose, kojibiose, sophorose, laminaribiose, turanose, isomaltulose, melibiose and combinations of two or more thereof. More preferably the disaccharide(s) is(are) selected from lactulose, lactose, maltose, isomaltose. More preferably the disaccharide comprises lactulose, lactose, maltose. Most preferably the disaccharide consists essentially of maltose.

Preferably the oligosaccharide (a saccharide polymer which typically contains from 3 to 10 monosaccharides) and/or polysaccharide (a saccharide polymer which typically contains more than 10 monosaccharides) is (are) selected from mannan-oligosaccharides, fructo-oligosaccharides, gluco-oligosaccharides, dextrin, maltodextrin, dextran, polydextrose, glucomannan, galactomannan, glucan, cellulose, hemi-cellulose, pectin or the like.

The composition of carbohydrate(s) (B) may preferably comprise, essentially consist of or consist of monosaccharides and/or disaccharides, preferably selected from glucose, mannose, rhamnose, fructose, maltose, isomaltose, maltulose, mannobiose and lactose.

The amounts of carbohydrate in the composition of carbohydrate(s) (B) as disclosed above apply to any of the compositions of carbohydrate(s) (B) according to the invention.

### 3. Mixing in the Aqueous Medium

The aqueous medium preferably comprises water, preferably essentially consists of water, more preferably consists of water.

The aqueous medium may also comprise ethanol and/or isopropanol.

The composition of protein(s) (A) and the composition of carbohydrate(s) (B) are either added together to an aqueous medium and mixed to prepare a composition (C) or they are individually dissolved/suspended in separate aqueous media, which are then mixed together to prepare a composition (C).

The composition (C) can be in the form of a suspension or a solution.

The mixing can occur in a micro-mixer or any other mixing apparatus.

The preferred weight ratio of composition of protein(s) (A) to composition of carbohydrate(s) (B) in the composition (C) ranges from 1:10 to 10:1, preferably 1:5 to 5:1, more preferably from 1:1 to 5:1, most preferably around 1:1.

Without being bound by theory, it is known that the most reactive amino acids towards modification with carbohydrates are the lysine amino acid residues in the protein (although these are not the only sites that react with the carbohydrate(s)). Thus the amount of carbohydrate to be added depends on the number of lysine amino acid residues in the protein and the desired degree of modification (glycation).

It has been observed that a 1:1 weight ratio of protein to carbohydrate results in the highest degree of modification (glycation), when glycating wheat proteins with glucose, in particular soluble wheat proteins with glucose.

### 4. pH Adjustment

It has surprisingly been found that the preferred pH of the composition (C) is somewhere between 7 and 8, although protein modification was also observed at a pH below 7 or above 8.

Preferably the pH of the composition (C) is from 6 to 9, more preferably from 6.5 to 9, even more preferably from 7 to 8.5, most preferably from 7 to about 8.

The pH of the composition (C) can be adjusted with any acid or base, as needed. The skilled person will know how much acid or base to add in order to arrive at the preferred pH range. Typically (but not exclusively), the starting materials have a more acidic pH and thus require base (e g ammonia, sodium hydroxide etc.) to increase the pH to the preferred pH range for the protein modification to occur.

According to an example, the pH can be adjusted with sodium hydroxide to reach the preferred pH ranges.

### 5. The Microdevice

Preferably, the microdevice comprises micro-heat exchanger(s) and/or micro-reactor(s), and optionally micro-mixer(s), and is suitable for the reaction of protein with carbohydrate, preferably in less than 150 seconds.

By microdevice it is meant herein to exclude microemulsions, which are disclosed in the literature as a kind of “microreactor” for food applications. By microdevice it is also meant herein to exclude open tubular capillary electrochromatography.

Microdevices are usually defined as miniaturized reaction vessels fabricated at least partially, by methods of microtechnology and precision engineering. The characteristics dimensions of the internal structure of microdevice fluid channels can vary substantially, but typically range from the sub-micrometer to the sub-millimeter range. The microdevice comprises micro-heat exchanger(s) and/or micro-reactor(s). The microdevice/microreactor/micro-heat exchanger have a capillary internal diameter of 1 mm or less, preferably less than 0.9 mm, 0.8 mm, 0.75 mm, 0.6 mm and less than 0.55 mm More preferably the microdevice and microreactor have a capillary diameter of from 0.1 mm, 0.2 mm, 0.3 mm, or 0.4 mm up to 0.6 mm, 0.75 mm, 0.8 mm, 0.9 mm, or 1 mm.

The microdevice/microreactor are often, but not necessarily, designed with microchannel architecture. These structures contain a large number of channels and each microchannel is used to convert a small amount of material. Free microstructure shapes, not forming dedicated channels, are also possible.

The benefits of miniaturized systems, designed with dimensions similar to microdevices/microreactors, compared to a large scale process include but are not limited to: that a large scale batch process can be replaced by a continuous flow process, smaller devices need less space, fewer materials, less energy and often shorter response times and system performance is enhanced by decreasing the component size, which allows integration of a multitude of small functional elements.

Typical thickness of the fluid layer in a microreactor can be set to few tens of micrometers (for example from about 10 to about 1000 μm) in which diffusion plays a major role in the mass/heat transfer process.

The micromixer is a static or kinetic micromixer, a diffusion micromixer, a cyclone-type micromixer, a multi-lamination micromixer, a focus micromixer or a split-and-recombine micromixer.

A static micro mixer is any type of micromixer in which the mixing of two or more fluids is performed by diffusion and optionally enhanced by transfer from laminar flow regime into transitional or turbulent flow regime such as described in EP 0 857 080.

A kinetic micromixer is a micromixer in which specially designed inlays produce a mixing by artificially eddies, or in which the mixing of two or more fluids is enhanced by applying kinetic energy to the fluids (e.g. stirring, high pressure, pressure pulses, high flow velocity, nozzle release).

A diffusion micromixer is a mixer of the static type, in which the fluids are ducted in that way, that the distance between the single fluids is in the range of the diffusion coefficients at the process parameters. In most cases, diffusion micromixers are taking advantage of multi-lamination of fluids such as described in EP 1 674 152, EP 1 674 150 and EP 1 187 671. A cyclone-type micromixer is a micromixer based on the rotational mixing of two or more fluids, which are inserted in a asymptotic or non-asymptotic way into a mixing chamber, providing rotational speed of each fluid flow which is also disclosed in EP 1 674 152.

A multi-lamination micromixer is a microstructure device where the single fluid streams are ducted very close to each other in lamination sheets or streams, to reduce the diffusion distance as it is disclosed in EP 1 674 152, EP 1 674 150, and EP 1 187 671.

A focus micromixer is a kinetic mixer in which fluid streams are focused into a dense meeting point to be mixed by kinetic energy and turbulence. A split-and-recombine micromixer is a micromixer where single fluid streams are split up by mechanical or non-tactile forces (e.g. electrical fields, magnetic fields, gas flow), changed in direction and position and recombined by, at least, doubling the number of sub-streams to increase the diffusion area.

The micro heat exchanger is a cross flow micro heat exchanger, counter-current flow micro heat exchanger, co-current flow micro heat exchanger or an electrically powered parallel flow micro heat exchanger and/or microreactors suitable for the modification of protein. A cross flow micro heat exchanger is a miniaturized plate heat exchanger in which the single fluid streams are ducted in a crosswise matter as is disclosed in EP 1 046 867.

A counter-current flow micro heat exchanger is a miniaturized plate heat exchanger in which the single fluid streams are ducted in a way that the inlets as well as the outlets of both fluids are in opposite direction to each other and therefore the fluid streams are running against each other, which is also described in EP 1 046 867.

A co-current flow micro heat exchanger is a miniaturized plate heat exchanger in which the single fluid streams are ducted in a way that the inlets as well as the outlets of both fluids are at the same direction of the device to each other and, therefore, the fluid streams are running in parallel which is described in EP 1 046 867.

An electrically powered parallel flow micro heat exchanger is a miniaturized heat exchanger where the heating or cooling energy is given by electrical elements (resistor heater cartridges, Peltier-Elements) such as described in e.g. EP 1 046 867, EP 1 402 589, EP 1 402 589.

The microreactor suitable for the modification of protein is a microchannel device, possibly integrated with at least a membrane, porous sidewalls or micro separation nozzle elements. Alternative solutions are provided by Kreido's microreactor that possesses a moving part, which in their case is the internal cylinder as is described in e.g. EP 1 866 066.

A microchannel device integrated with a membrane is preferably in the range of 1 to 2000 μm wide, 1 to 2000 μm deep and in direct contact with the membrane, which forms at least one side wall of the channel. The membrane can be a polymer, metal or ceramic membrane with pore sizes according to the process needs, ranging from some nanometer to the micrometer level. Porous sidewalls have pores of the same specifications than the membranes or micro separation nozzle elements suitable for the desired process, preferably in the range of some nanometer up to 1 mm diameter. The current invention relates to a process wherein the microdevice is applied at sub-atmospheric pressure, atmospheric pressure or elevated pressure, in the range from very low pressures in the ultra-high vacuum range (almost 0 bar) to 1000 bar.

### 6. Reaction Conditions in the Microdevice

Optionally, before adding (either by pumping or injecting) the composition (C) through the microdevice, the composition (C) can be heated by using a micro-heat-exchanger and/or microwaves or any other suitable heating device.

Once added into the microdevice, the temperature of the reaction in step (d) is preferably 50 to 120° C., preferably 60 to 110° C., more preferably 70 to 100° C. and most preferably 80 to 95° C.

Without being bound to theory, temperature may have two distinct effects on Maillard reactions between proteins and carbohydrates depending on the reaction stage. An increase in temperature can lead to increased, desirable glycation in the early stage of the Maillard reaction (Cheison, Josten, & Kulozik, 2013; Chen, Liang, Liu, Labuza, & Zhou, 2012; Naranjo, Malec, & Vigo, 1998).

In later stages, increased temperature can influence the reaction mechanism and consequently lead to the formation of various intermediary and end products such as melanoidin structures (Benzingpurdie, Ripmeester, & Ratcliffe, 1985) and others which may cause substantially coloring. Thus, late stage Maillard reactions leading to advanced Maillard products and substantial coloring can for instance be avoided by using lower temperatures at a later stage of the microdevice.

Thus, when using multiple passes, zones or microreactors, a first pass or zone in a microdevice/microreactor preferably can have a higher reaction temperature than a second pass in the same or second microreactor/microreactor (or than a second zone in the same microdevice/microreactor).

The execution of glycation reactions in a microdevice/microreactor requires that the reactants are present in a liquid or aqueous medium. Initially, the inventors expected that the viscosity of the medium might cause a high pressure drop in the channels demanding an elevated energy input to convey the product through them. Surprisingly, the inventors found that the product could be easily conveyed in the aqueous medium through the channels without any pressure drop. Also any undissolved particles above a certain mesh size could create blockages of the channel(s). Surprisingly, no blockages were observed, as shown in the examples below. Too much aqueous medium was also thought to hinder the reaction dynamics and the economics of the process due to potentially unacceptable energy demand to remove excess water to drive the reaction to a higher yield. Again, the inventors were surprised that up to 20% glycation could be observed. Furthermore, the reaction could be surprisingly carried out to such degrees of glycation without needing to increase the pH to above pH 8. Without being bound by theory it is thought that a too alkaline pH, e.g. pH 11 as observed in batch reactions as resulting in the highest degrees of glycation, might cause additional byproduct formation such as hydroxy-methylfurfural (HMF). By carrying out the reaction at only slightly alkaline pH e.g. 7 to 8, not only can significant HMF production be avoided, but surprisingly also more advanced stage Maillard reactions can be avoided.

Since the yield and degree of modification (e.g. glycation) is a function of reaction time, the desired degree of protein modification can be obtained by quenching the reaction at the appropriate time. It has been seen that by applying a microdevice the reaction time which usually takes several hours or even days (depending on solution or dry state) can be reduced to a reaction time of less than 150 seconds, preferably less than 120 seconds, more preferably less than 110 seconds, even more preferably less than 100 seconds, most preferably from 0.1, 0.5 or 1 to around 90, 80, 70, 60, 50, 40, 30, 20 or 10 seconds, e.g. 0.1 to 90 seconds or 1 to 90 seconds or 0.5 to 50 seconds etc. Thus the microdevice can be used to control the reaction time and avoid undesirable Maillard reactions and coloring.

Furthermore the current invention relates to a process wherein the modified protein after leaving the microdevice is quenched. Quenching might include the addition of adding water, with a base: caustic soda, potassium hydroxide but also amines; at elevated temperature in the range of 50 to 150° C. to ensure the modified protein does not solidify or become too viscous in a micro-mixer, micro heat exchanger, a microstructure evaporator or a microstructure steam dryer. A micro heat exchanger is a cross flow micro heat exchanger, counter-current flow micro heat exchanger, co-current flow micro heat exchanger or an electrically powered parallel flow micro heat exchanger and/or microreactors suitable for the modification of protein, according to the definitions given above. A microstructure evaporator is a micro heat exchanger suitable and/or specially designed for evaporation of liquids. Examples are given in e.g. EP 1 402 589.

A microstructure steam dryer is a microstructure evaporator according to the given explanation, used to dry a steam flow, e.g. to obtain crystallization of solid contents in the steam.

The process according to the invention may further comprise a step (e) for adding the composition of modified protein(s) (D), optionally mixed with a further composition of carbohydrate(s) (B′) (which can be same or different from the composition (B)), back into the same microdevice and/or into a second microdevice to increase the degree of modification of the protein. Through this recycle the yield and/or the degree of modification (e.g. glycation) of the modified protein can be further increased.

The microreactor for the recycle might be the same as used before in the process or a set of multiple (at least two or more) sequential microreactors can be applied.

Before collecting the composition of modified proteins(s) (D), it can be cooled by using a micro heat exchanger.

The current invention also relates to a process for preparing glycated wheat and/or soybean and/or whey protein(s) and said process comprises the following steps:
a) Mixing a composition comprising wheat and/or soybean and/or whey protein(s) (A) and a composition comprising (or essential consisting of or consisting of) glucose (B) to prepare a composition (C) in an aqueous medium;
b) Optionally adjusting the pH of the composition (C), preferably adjusting the pH of the composition (C) to a pH of from 6 to 9, more preferably from 6.5 to 9, even more preferably from 7 to 8.5, most preferably from 7 to 8;
c) Adding the composition (C) into a microdevice;
d) Reacting the wheat and/or soybean and/or whey protein(s) with the glucose in the microdevice to obtain a composition comprising glycated wheat and/or soybean and/or whey proteins (D).

The composition of modified wheat and/or soybean and/or whey protein(s) (D) can be recovered.

All preferred features disclosed herein can also be applied and combined with this process.

Furthermore, the formation of degradation products and advanced Maillard reaction products such as furans, furfural and 5-hydroxymethyl furfural (5HMF) can be surprisingly significantly reduced by using the process as claimed.

The recovered modified protein can be used as is or may be further purified by chromatographical treatment or hydrogenation of residual reducing sugars (for example monosaccharides and certain disaccharides that have a reducing end) that may have an effect on the taste and color of the final product.

The current invention further relates to the use of a microdevice for the modification of protein with carbohydrate(s), preferably for the glycation of proteins, most preferably for the glycation of wheat and/or soybean and/or whey protein(s). This includes for instance the use of a microdevice for the modification of protein(s) with carbohydrate(s), preferably for the glycation of protein with a mono-, di-, oligo- or polysaccharide(s), wherein the microdevice comprises micro-heat exchangers and/or micro-reactors and optionally micro-mixer(s), and is suitable for the reaction of protein with carbohydrate, preferably with a reaction duration of less than 150 seconds, and preferably at a reaction temperature of at least 60° C. For the avoidance of doubt, any embodiments, features, elements disclosed herein are equally applicable to the use of the microdevice according to the invention.

Furthermore, the current invention relates to the use of an arrangement of microdevices allowing a single-pass-through or a multi-pass-through of injected composition through the microdevice, a re-mix of the collected modified protein with the initial composition for a multi-pass-through or a complete multi-pass-through for the composition. Other arrangements might include: a) microdevice-evaporator-microdevice (same as first one or different) and b) several iterations of a) and c) microdevice-evaporator with recirculation into same microdevice.

The process may thus further comprise a step wherein the composition of modified protein(s) (D) is added back into the same microdevice and/or into a second microdevice to increase the degree of modification of the protein. The composition of modified protein(s) (D) can also be mixed with a further composition of carbohydrate(s) (B′), which can be same or different from the composition (B), before being added back into the same microdevice and/or into a second microdevice to increase the degree of modification of the protein.

### 7. Composition of Modified Protein(s) (D)

The composition of modified protein(s) (D) obtained from step (d) have an increased amount of modified proteins compared to the protein(s) in composition (A).

Without being bound by theory, it is thought that the carbohydrate successfully reacts with certain aminoacid residues in the protein so as to modify them. This modification is preferably “glycation” i.e. when the composition of carbohydrate(s) (B) comprises, essentially consists of or consists of mono- and/or disaccharides, preferably one or more of rhamnose, glucose, mannose, fructose, maltose, isomaltose, maltulose, mannobiose and lactose.

The current invention also relates to wheat protein glycated with carbohydrate(s), preferably one or more of mono-, di-, oligo- or polysaccharide(s). Preferably the invention relates to a wheat protein glycated with one or more of glucose, mannose, galactose, rhamnose, fructose, maltose, isomaltose, maltulose, mannobiose and lactose, more preferably with glucose. The glycated wheat protein has improved physical properties allowing it be used in many food, feed, personal care, cosmetic, pharmaceutical, paper and corrugated board products. For instance, the inventors have shown that glycated wheat protein has a better foam stability (see Example 2 below).

The degree of modification, including the degree of glycation, can be measured using the principles of the OPA method (“Improved method for determining food protein degree of hydrolysis”, P. M. Nielsen, D. Petersen and C. Dambmann J of Food Sciences, 2001, Vol. 66, no 5, p. 642-6465). The OPA method is used to measure the number of free α and ε-amino acids. The number of free α and ε-amino acids increases as a result of hydrolysis. However, the OPA method can also be adapted to measure the degree of glycation of proteins, as glycation decreases the number of free α and ε-amino acids (see method below).

### 8. Use of the Composition Obtainable According to the Process of the Invention

The compositions of modified protein (D) or (D′) obtainable and obtained according to the process of the current invention can be used in food, feed, personal care, cosmetic, pharmaceutical, paper and corrugating board products together with at least one other ingredient. Before being used in such applications, the compositions of modified protein(s) (D) or (D′) can be purified according to any known means for purifying proteins.

The invention also relates to a process for preparing a food, feed, personal care, cosmetic, pharmaceutical, paper or corrugated board product comprising the process for preparing a composition comprising modified protein(s) (D) as stated above and the step of combining the composition comprising modified protein(s) (D) with at least one other ingredient.

Any embodiments, features, elements disclosed herein are equally applicable to the use of the composition (D) of the invention.

The invention will hereunder be illustrated in the form of a series of examples.

### 9. Methods of Measurement

### 9.1. OPA Method to Determine the Extent of Glycation

This method allows for measurement of loss of free amine groups in glycated samples via the reaction between this free amino groups and OPA reagent, producing a compound which shows absorbance at 340 nm. The degree of glycation (or degree of protein modification) is therefore calculated by measuring the ratio between the amount of free amine groups in the glycated samples and the non-glycated samples.

In detail, the ortho-phthaldialdehyde (OPA) reagent was prepared as described by Nielsen et al. (2001), with some modifications: 0.160 g of OPA, 4 ml of methanol, 100 ml of 0.1 M di-sodium tetraborate (Borax) solution, 0.8 g of 2-dimethylaminoethanethiol hydrochloride (DMAC) and 20 ml of 10% (w/w) Sodium Dodecyl Sulphate (SDS) solution, previously made, were dissolved in a 200 ml volumetric flask, completing with ultrapure water (obtained from RiOs™ Water Purification System (Merck)). Furthermore, a calibration curve of the spectrophotometer with different dilutions of L-lysine was made to calculate the NH2 concentration of the products, based on dry substance.

To start with the test, each product was diluted in 15 ml plastic tubes until a level of from 0.5 to 3 wt % (dry weight). The plastic tubes were vortexed to make sure the solution was homogeneous. Then, a blank sample was prepared by adding 30 μl of ultrapure water+3 ml of OPA reagent to a 35 ml plastic disposable cuvette. The rest of the samples were prepared in duplicate following the same procedure but substituting the ultrapure water by the different product dilutions. After filling each cuvette, they were covered with Parafilm and mixed by reversing them several times.

After filling a series of 6 cuvettes, the timer was set for 10 minutes to wait for the absorption readout to become stable. Finally, the absorbance was measured at a wavelength of 340 nm with a spectrophotometer model GENESYS™ 10S UV-Vis (Thermo Fisher Scientific™) The degree of glycation was calculated as the ratio of the amount of free α and ε-amino acids in the product (Composition (D)) to the amount of free α and ε-amino acids in the starting raw material (Composition (A)), using Equation (2).

$$DS = 100 - ( \frac{{NH}_{2 product}}{{NH}_{2 reference}} \cdot 100 ) \qquad ( 1 )$$

The features disclosed in the description including the Examples below, or the following claims, or the accompanying drawings, expressed in their specific forms or in terms of a means for performing the disclosed function, or a method or process for attaining the disclosed result, as appropriate, may be used separately or in any combination for realizing the invention in any of its forms.

Although certain example embodiments of the invention have been described, the scope of the appended claims is not intended to be limited solely to these embodiments. The claims are to be construed literally, purposively, and/or to encompass equivalents.

### EXAMPLES

### Example 1: Glycation of Solubilized Hydrolyzed Wheat Protein (sHWP) with Glucose in a Microreactor

### The Reactants:

Appropriate amounts of protein compositions (A) of sHWP having the properties as provided in Table 1 were dissolved in the appropriate amount of preheated deionized water (40° C.) and continuously stirred for 1 hour until the solid material had dissolved in order to obtain samples having 10 wt %, 20 wt % or 30 wt % of the protein composition (A).

The sHWP used in these examples can be obtained from hydrolysed wheat protein according to EP 2117338B1. The total protein content in dry matter of the sHWP was about 79% w/w. It had a degree of hydrolysis of about 6% (determined using the OPA method).

TABLE 1
Properties of Protein Composition (A) of sHWP

| | Properties of sHWP |
|---|---|
| Dry substance (wt %) | 96.5 |
| pH (at 10% dry substance) | 6.0 |
| N factor | 5.70 |
| Protein content (wt % on dry | 79.7 |
| substance) | |

Appropriate amounts of carbohydrate compositions (B) of glucose (C*Dex 02042, a commercially available crystalline α-D-glucose (dextrose) monohydrate with a dry substance of 91.5 wt % from Cargill) were dissolved in the appropriate amounts of preheated deionized water (40° C.) and continuously stirred for 1 hour until the solid material had dissolved in order to obtain samples having 10 wt %, 20 wt % or 30 wt % of the carbohydrate composition (B).

Appropriate amounts of the aqueous samples comprising Compositions A and Compositions B were mixed and stirred for a further 30 minutes in order to prepare the required samples of Compositions (C) in an aqueous medium having the desired overall dry substance and weight ratio of Protein Composition (A) to Protein Composition (B) as disclosed in Table 2 below.

The native pH-value of Composition (C) at 40° C. was between 5.3 and 6.2.

TABLE 2
Preparation of samples according to Compositions (C)

| | Weight ratio of the | |
|---|---|---|
| | composition (A) comprising | |
| | sHWP to composition (B) | Name of |
| Dry Substance (%) | comprising dextrose | run |
| 10 | 1:1 | RUN 1 |
| | 1:1 | RUN 2 |
| | 1:1 | RUN 3 |
| | 2:1 | RUN 4 |
| | 2:1 | RUN 5 |
| | 2:1 | RUN 6 |
| | 5:1 | RUN 7 |
| | 5:1 | RUN 8 |
| 20 | 1:1 | RUN 9 |
| 30 | 1:1 | RUN 10 |
| | 1:1 | RUN 11 |
| | 1:1 | RUN 12 |
| | 1:1 | RUN 13 |
| | 1:1 | RUN 14 |
| | 1:1 | RUN 15 |
| | 1:1 | RUN 16 |
| | 1:1 | RUN 17 |
| | 1:1 | RUN 18 |
| | 1:1 | RUN 19 |
| | 1:1 | RUN 20 |
| | 1:1 | RUN 21 |
| | 1:1 | RUN 22 |
| | 1:1 | RUN 23 |
| | 1:1 | RUN 24 |
| | 1:1 | RUN 25 |
| | 1:1 | RUN 26 |
| | 1:1 | RUN 27 |
| | 1:1 | RUN 28 |

### The Microdevice:

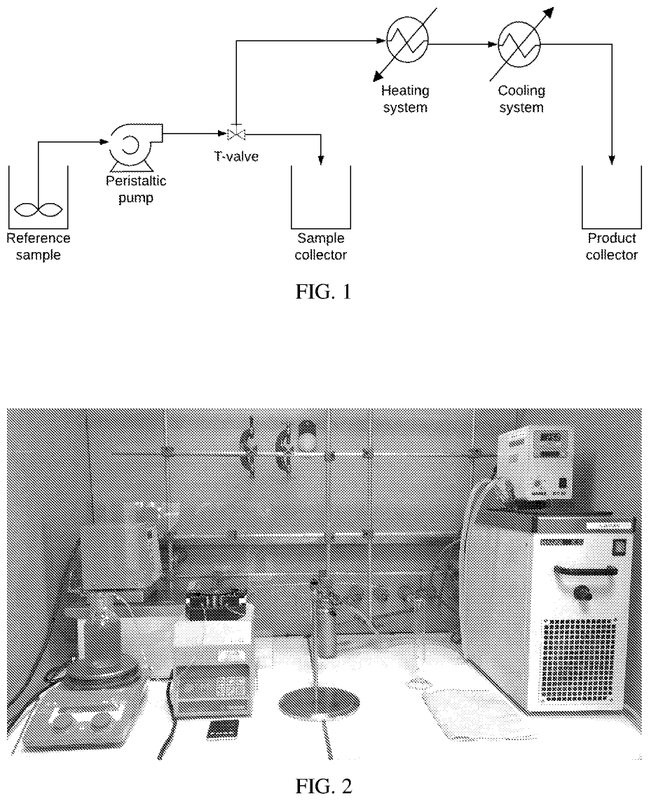

A microdevice was set up as shown in FIGS. 1 and 2 composed of the following elements:

(1) a magnetic stirrer, (2) a peristaltic pump (Minipuls 3, Gilson), (3) a heating circulator (MC4, Julabo) filled with heat-resistant oil, (4) a water bath (DC50, Haake) connected to a closed cooling system made in-house, (5) stainless steel capillaries, (6) a T-valve, (7) a glass bottle for inlet sample material and (8) a glass cylinder to collect outlet products.

Its operation principle is as follows: the initial sample is pumped through the capillary system, from which a fixed length is immersed in the oil circulator in order to be subjected to a heat treatment (90° C.) for specific times. Subsequently, the material is cooled inside the capillary system through a water bath that supplies cooling water to an in-house made close tank. Finally, the product is collected to be further analysed.

In a microreactor, a larger production can be attained by either increasing the reaction volume (in other words, the length of the microcapillary) or by adding more microreactors in parallel under the same conditions (Ramanjaneyulu et al., 2018). In this case, to reach the desired reaction volume, the capillary length was calculated assuming a fixed radius of 0.50 mm Thereupon, the main specifications of the microreactor, such as microcapillary volume, radius and length, are shown in Table 3.

TABLE 3
Main specifications of the microreactor.

| Specification | Symbol | Units | Value |
|---|---|---|---|
| Microcapillary volume | V_{cylinder} | mm³ | 597 |
| Microcapillary radius | R | mm | 0.50 |
| Microcapillary length | L | mm | 760 |

Considering the previously specified volume of the capillary which is immersed in the oil bath, and assuming a desired constant flowrate of 1.2 g/min and a density similar to water, the residence time of the reference sample subjected to glycation, in seconds, could be calculated according to Equation (2):

$$τ = \qquad ( 2 )$$

$$\frac{{V}_{cylinder}}{{v}_{0}} = \frac{( π {R}^{2} L ) {ρ}_{w}}{{F}_{pump}} = \frac{{π ( 0.5 mm )}^{2} ( 760 mm ) ( 0.001 \frac{g}{{mm}^{3}} )}{( 1.2 \frac{g}{min} ) ( \frac{1 min}{60 s} )} = 29.85 s$$

Therefore, residence times of subsequent trials could be calculated by substituting the pump flowrate in the previous equation.

### The Reaction Conditions:

A large series of microreactor experiments with Compositions (C) of sHWP and glucose were performed, to define the optimal processing conditions, by varying:
the percentage of dry substance (from 10 to 30% w/w),
the protein composition (A) to carbohydrate composition (B) weight ratio (from 1:1 to 5:1),
the pH (from 5.2 to 9.0) and
the residence time (from 60 to 270 s), by either changing the flow rate (g/min) in the capillary or the number of passes (from 1 to 3) through the system.
   All conditions tested are presented in Table 4 below.

TABLE 4
Processing conditions tested in the microreactor trials.

| | | Weight ratio of the | | |
|---|---|---|---|---|
| | | composition (A) | | |
| | Dry | comprising sHWP to | | |
| | substance | composition (B) | Target | |
| Mixture | (%) | comprising dextrose | pH | Target residence time (s) |
| sHWP:dex* | 10 | 0:1 | 5 | 30, 60, 90 (1 pass) |
| | 20 | 1:0 | 6 | 60, 120, 180 (1, 2 and 3 passes) |
| | 30 | 1:1 | 7 | 90, 180, 270 (1, 2 and 3 passes) |
| | | 2:1 | 8 | 120 (1 pass) |
| | | 5:1 | | |

*dex means dextrose i.e. glucose

Where required, the pH adjustments of Composition (C) were conducted using a solution of 5% (w/w) sodium hydroxide.

After turning on all the individual elements of the system, the heating circulator and the cooling system temperatures were set at 90° C. and 25° C., respectively. Then, the desired pump speed was selected. It was essential to check that the valve was opened for the product collector and also that the microreactor tubing was indeed immersed in the oil bath.

When the inlet tubing was placed inside the reference sample bottle, which had to be constantly stirring at 120 rpm, the pump was started. At this point, the system had to be rinsed with the initial solution for at least 5 minutes in order to ensure the tubing had been completely homogenized.

Subsequently, the product collector had to be placed in the outlet tubing and, at the same time, the stopwatch had to be started. When the trial had been completed, the pump was stopped and the glycated product was stored in the freezer, at −18° C., to avoid microbial growth. At this point, several tasks had to be completed with the aim of collecting accurate data to process at a later stage.

First of all, the amount of dry substance of each reference sample and product with a MA 150 (Sartorius) moisture analyzer was measured to check no losses of material had occurred during the trial.

The timing of the trials and the weight of the samples before and after was, as well, used to calculate the exact flowrate and the exact residence time of the sample inside the microreactor. Additionally, measuring the pH of the reference sample, both before and after the trial, and the pH of each obtained product was key to monitor the influence of pH in the Maillard reaction.

### The Results:

The degree of glycation (protein modification) was determined according to the OPA method described above.

In the production of protein-polysaccharides conjugates, the rate, extent and course of the Maillard reaction are influenced by several factors, including nature of the reactants, protein to carbohydrate ratio, temperature, time, pH and water activity or relative humidity.

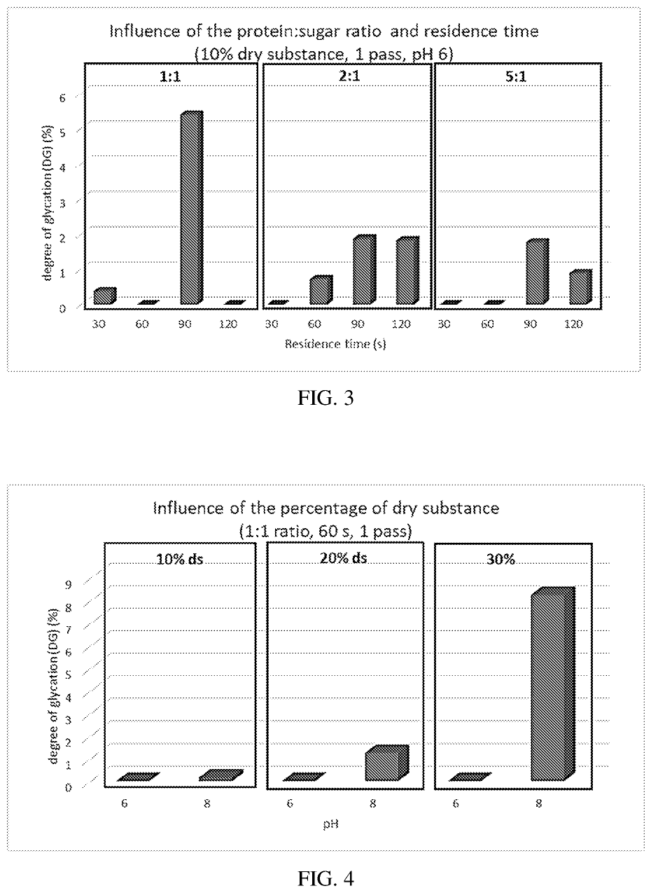

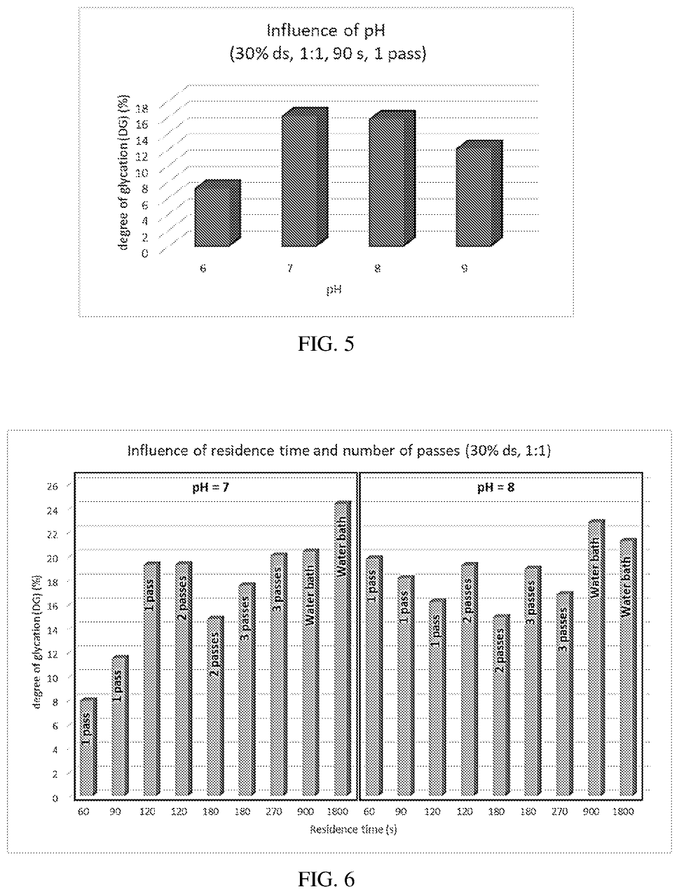

Results in terms of degree of glycation are shown in Table 5 below and in FIGS. 3 to 6.

TABLE 5
Results

| | | Weight ratio of the | | | | |
|---|---|---|---|---|---|---|
| | | composition (A) | | | | Degree |
| Dry | | comprising sHWP to | | | Target | of |
| Substance | Name of | composition (B) | Initial | Final | residence time | glycation |
| (%) | run | comprising dextrose | PH | PH | (s) | (%) |
| 10 | RUN 1 | 1:1 | 5.71 | 5.75 | 30 (1 pass) | 0.38 |
| | RUN 2 | 1:1 | 8.05 | 8.00 | 60 (1 pass) | 0.16 |
| | RUN 3 | 1:1 | 5.83 | 5.84 | 90 (1 pass) | 5.40 |
| | RUN 4 | 2:1 | 5.86 | 5.85 | 60 (1 pass) | 0.72 |
| | RUN 5 | 2:1 | 5.86 | 5.85 | 90 (1 pass) | 1.88 |
| | RUN 6 | 2:1 | 5.86 | 5.86 | 120 (1 pass) | 1.83 |
| | RUN 7 | 5:1 | 5.89 | 5.89 | 90 (1 pass) | 1.77 |
| | RUN 8 | 5:1 | 5.89 | 5.89 | 120 (1 pass) | 0.89 |
| 20 | RUN 9 | 1:1 | 7.91 | 7.86 | 60 (1 pass) | 1.24 |
| 30 | RUN 10 | 1:1 | 7.90 | 7.73 | 60 (1 pass) | 8.25 |
| | RUN 11 | 1:1 | 5.65 | 5.64 | 90 (1 pass) | 7.22 |
| | RUN 12 | 1:1 | 6.79 | 6.52 | 90 (1 pass) | 16.17 |
| | RUN 13 | 1:1 | 7.98 | 7.69 | 90 (1 pass) | 15.79 |
| | RUN 14 | 1:1 | 8.96 | 8.72 | 90 (1 pass) | 12.16 |
| | RUN 15 | 1:1 | 6.61 | 6.51 | 120 (1 pass) | 19.23 |
| | RUN 16 | 1:1 | 76.61 | 6.54 | 60 (1 pass) | 7.92 |
| | RUN 17 | 1:1 | 6.61 | 6.54 | 120 (2 passes) | 19.25 |
| | RUN 18 | 1:1 | 6.61 | 6.54 | 180 (3 passes) | 17.50 |
| | RUN 19 | 1:1 | 6.63 | 6.58 | 90 (1 pass) | 11.46 |
| | RUN 20 | 1:1 | 6.63 | 6.58 | 180 (2 passes) | 7.91 |
| | RUN 21 | 1:1 | 6.63 | 6.58 | 270 (3 passes) | 20.00 |
| | RUN 22 | 1:1 | 7.848 | 7.60 | 90 (1 pass) | 18.12 |
| | RUN 23 | 1:1 | 7.84 | 7.54 | 180 (2 passes) | 14.87 |
| | RUN 24 | 1:1 | 7.84 | 7.52 | 270 (3 passes) | 16.76 |
| | RUN 25 | 1:1 | 8.01 | 7.81 | 120 (1 pass) | 16.16 |
| | RUN 26 | 1:1 | 8.01 | 7.89 | 60 (1 pass) | 19.74 |
| | RUN 27 | 1:1 | 8.01 | 7.81 | 120 (2 passes) | 19.18 |
| | RUN 28 | 1:1 | 8.01 | 7.74 | 180 (3 passes) | 18.90 |

The proteins could be successfully glycated at a much faster rate than using the conventional methods in a dry state or wet state, which take several hours or even days, whilst still achieving high rates of glycation.

The highest rate of glycation was observed when the composition (C) had:
a dry substance of about 30 wt %,
a 1:1 weight ratio of the protein composition to the glucose composition,
a pH of from 7 to 8 and
the residence time in the reactor was from 60 to 90 s.

### Example 2: Improved Foaming Properties of the Glycated sHWP

The foaming properties of the glycated sHWP were compared with the non-glycated sHWP, both as a simple mixture with dextrose (before injection into the microdevice) and with sHWP alone.

The experiments were first carried out with the glycation reaction in the microdevice as described above in Example 1 at a dry substance of 10 wt % with a target residence time of 60 seconds, except that the initial pH of the mixture (measured at pH 5.35) was not adjusted. Ratios of sHWP composition to dextrose composition of 1:1 and 5:1 were tested. The obtained product was freeze-dried down to an average moisture content of about 4 wt %, measured with a Sartorius 150 MA at 90° C. for 15 minutes.

The resulting foam volume capacity of the dried product when mixed with water (at a concentration of 1% w/v of protein) was measured in a cylindrical measuring flask. The resulting foam volume capacity was between 430 and 460 ml foam per g of protein at time zero (T0).

Foam stability was measured by comparing the volume of foam at time zero (T0) with the volume of foam remaining at 30 minutes (T30), without any agitation at ambient temperature. The percent of foam remaining after 30 minutes is shown in the table below.

| | | Foam Stability |
|---|---|---|
| Description of sample | pH | (after 30 min) % |
| sHWP alone* | 5.35 | 5.32 |
| 1:1 mixture of sHWP composition to dextrose | 5.35 | 2.63 |
| composition (before glycation reaction in the | | |
| microdevice) | | |
| 1:1 sHWP composition to dextrose composition | 5.35 | 7.41 |
| (after glycation reaction in the microdevice) | | |
| 5:1 mixture of sHWP composition to dextrose | 5.35 | 2.63 |
| composition (before glycation reaction in the | | |
| microdevice) | | |
| 5:1 sHWP composition to dextrose composition | 5.35 | 33.33 |
| (after glycation reaction in the microdevice) | | |

*sHWP (described in Example 1) at a concentration of 1% w/v of protein in water to measure foam capacity and foam stability.

It can be seen that foam stability increases after glycation in the microdevice and also in comparison to sHWP alone.

The above was then repeated, except that the pH of the mixture was adjusted from pH 5.35 to pH 8.0 using an 5 wt % NaOH solution as also described in Example 1.

| | | Foam Stability |
|---|---|---|
| Description of sample | pH | (after 30 min) % |
| sHWP alone | 8.0 | 1.75 |
| 1:1 mixture of sHWP composition to dextrose | 8.0 | 2.54 |
| composition (before glycation reaction in the | | |
| microdevice) | | |
| 1:1 sHWP composition to dextrose composition | 8.0 | 3.51 |
| (after glycation reaction in the microdevice) | | |
| 5:1 mixture of sHWP composition to dextrose | 8.0 | 9.48 |
| composition (before glycation reaction in the | | |
| microdevice) | | |
| 5:1 sHWP composition to dextrose composition | 8.0 | 25.42 |
| (after glycation reaction in the microdevice) | | |

Again, it can be seen that foam stability increases after glycation in the microdevice also at pH 8.0 and also in comparison to sHWP alone at the same adjusted pH of 8.0.

### Comparative Example 1—Batch Reactions to Glycate sHWP

To investigate the feasibility of an sHWP glycation reaction in an aqueous solution in a batch process an Eppendorf ThermoMixer® was used. Different solutions of the protein/carbohydrate mixture, at 5% solids content, 1:1 protein to sugar ratio, and pH 6-11, were placed in small vials of 2 ml volume. These test tubes were placed in the heated thermo-block of the ThermoMixer® to heat the mixture to the desired temperature while shaking the tubes at a speed of ca. 800 min⁻¹. The small sample quantity allowed a fast heating of the solution. The overall residence time was limited to max. 30 minutes. Afterwards the reaction was stopped by placing the vials in an ice bath.

The following conditions were used:

| | Ratio sHWP | | | |
|---|---|---|---|---|
| Solids | composition to | | | Reaction |
| content % | glucose | | Reaction time | temperature |
| (w/w) | composition | pH | (min) | (° C.) |
| 5 | 1 : 1 | 6, 7, 9, 11 | 1, 5, 10, 15, | 90 |
| | | | 20, 25, 30 | |

The feasibility of the glycation reaction in aqueous solution was evaluated on small batch scale using a 2 ml sample size.

The measurement of the degree of substitution showed no glycation occurring at pH 6 and 7 for any length of residence time up to 30 minutes. Only at much more alkaline conditions, i.e. pH 9 and 11, did some reactions occur. The highest degree of lysin functionalization was found at pH 11 and 30 minutes residence time showing that the glycation reaction is feasible in aqueous solution, but requiring high pH and long reaction times.

A potential explanation could be that Maillard reactions are time critical, i.e. the longer the reaction time under certain conditions the more advanced the reaction proceeds, but also to more complex products. The OPA method determines the quantity of remaining amino groups regardless to which stage the Maillard reaction has evolved. Without being bound by theory, it is thought that thanks to the extremely short reactions times, the glycation reaction using microreactors yields only the very early Maillard products, i.e. the simple Amadori compounds. Therefore, the short reaction times are surprisingly favorable. Additionally a too alkaline pH, such as at pH 11, is not desirable either as this encourages the isomerization of glucose to fructose, which potentially could result in additional undesirable by-product formation, e.g. hydroxy-methylfurfural (HMF).

### Example 3: Glycation of Defatted Soy Flour (Prolia® FLR-100/90) with Glucose in a Microreactor

### The Reactants:

Method (a) for Preparing the Reactants—Protein Composition (A) was Centrifuged or Sieved First Before Mixing with Glucose Composition (B)

Appropriate amounts of protein compositions (A) comprising soy flour (SF) having the properties as provided in Table 6 were dissolved in the appropriate amount of deionized water at room temperature (ca. 23° C.) and stirred at a gradually increasing speed (400 rpm to 750 rpm) for 30 min until most of the solid material had dissolved in order to obtain samples having 10 wt % of the protein composition (A). As the SF solution after 30 min still contained some insoluble material the sample was centrifuged at 3500G for 20 minutes (Thermo Scientific™ Heraeus Labofuge 400) and the supernatant solution was used for further processing. An alternative method to centrifugation that was tested was filtration of the insoluble material via an appropriate screen, e.g. 100 μm mesh size.

TABLE 6
Properties of Protein Composition (A) of SF

| | soy flour (Prolia ® FLR-100/90) |
|---|---|
| Dry substance (wt %) | 94.0 |
| pH (at 10% dry substance) | 6.49 |
| N factor | 5.70 |
| Protein content (wt % on dry | 53.5 |
| substance) | |

Appropriate amounts of carbohydrate compositions (B) of glucose (C*Dex 02042, a commercially available crystalline α-D-glucose (dextrose) monohydrate with a dry substance of 91.5 wt % from Cargill) were dissolved in the appropriate amounts of preheated deionized water (40° C.) and continuously stirred for 1 hour until the solid material had dissolved in order to obtain samples having 10 wt % of the carbohydrate composition (B).

Appropriate amounts of the aqueous samples comprising Compositions A and Compositions B were mixed and stirred for a further 30 minutes in order to prepare the required samples of Compositions (C) in an aqueous medium having the desired overall dry substance and weight ratio of Protein Composition (A) to Protein Composition (B) as disclosed in Table 7 below.

### Method (b) for Preparing the Reactants—Composition (C) in Aqueous Medium was Centrifuged

Appropriate amounts of protein compositions (A) comprising soy flour (SF) having the properties as provided in Table 6 were dissolved in the appropriate amount of deionized water at room temperature (ca. 23° C.) and stirred at a gradually increasing speed (400 rpm to 750 rpm) for 30 min until most of the solid material had dissolved in order to obtain samples having 10 wt % of the protein composition (A).

Appropriate amounts of carbohydrate compositions (B) of glucose (C*Dex 02042, a commercially available crystalline α-D-glucose (dextrose) monohydrate with a dry substance of 91.5 wt % from Cargill) were dissolved in the appropriate amounts of preheated deionized water (40° C.) and continuously stirred for 1 hour until the solid material had dissolved in order to obtain samples having 10 wt % of the carbohydrate composition (B).

Appropriate amounts of the aqueous samples comprising Compositions A and Compositions B were mixed and stirred for a further 30 minutes in order to prepare the required samples of Compositions (C) in an aqueous medium having the desired overall dry substance and weight ratio of Protein Composition (A) to Protein Composition (B) as disclosed in Table 7 below.

As the Compositions (C) in aqueous medium after the 30 min of mixing still contained some insoluble material, the sample was centrifuged at 3500G for 20 minutes (Thermo Scientific™ Heraeus Labofuge 400) and the supernatant solution was used for further processing. (Note that this caused the dry substance content to drop to about 8.5%).

The native pH-value of all Compositions (C) at ambient temperature was between 6.5 and 6.7.

TABLE 7
Preparation of samples according to Compositions (C)

| | Weight ratio of the composition (A) | |
|---|---|---|
| Dry | comprising soy flour to composition | |
| Substance (%) | (B) comprising dextrose | Name* |
| 10 | 1:1 | Trial 1 (a)** |
| | 1:5 | Trial 2 (a)** |
| | 1:1 | Trial 3 (a) |
| | 1:1 | Trial 3 (b) |
| | 1:1 | Trial 4 (a) |

*the (a) and (b) in the trial names refer to reactants prepared according to methods (a) or (b) respectively
**in trials 1 and 2 SF sample was sieved via a 100pm sieve prior to mixing with dextrose solution, instead of centrifugation

### The Reaction Conditions:

A series of microreactor experiments using the trial set-up as described in example 1 with Compositions (C) of soy flour and glucose were performed applying following conditions:
the percentage of dry substance (10% w/w) (except for reactants prepared according to method (b), where the dry substance content was around 8.5% w/w),
the protein composition (A) to carbohydrate composition (B) weight ratio (from 1:1 to 1:5),
the pH (from 5.2 to 9.0) and
the residence time (10 and 30 s), by adjusting the flow rate (g/min) in the capillary
   All conditions tested are presented in Table 8 below.

TABLE 8
Processing conditions tested in the microreactor trials.

| | | Weight ratio of the | | |
|---|---|---|---|---|
| | | composition (A) | | |
| | Dry | comprising soy flour to | | |
| | substance | composition (B) | Target | Target residence |
| Mixture | (%) | comprising dextrose | pH | time (s) |
| SF:dex* | 10 | 1:1, 1:5 | 8 | 10 & 30 (one pass) |

*dex means dextrose i.e. glucose

Where required, the pH adjustments of Composition (C) were conducted using a solution of 5% (w/w) sodium hydroxide for alkaline pH and for any acidic pH adjustments 0.1 N hydrochloric acid was used.

The microreactor experiments were run in the same manner as in Example 1.

### The Results:

The degree of glycation (protein modification) was determined according to the OPA method described above.

In the production of protein-polysaccharides conjugates, the rate, extent and course of the Maillard reaction are influenced by several factors, including nature of the reactants, protein to carbohydrate ratio, temperature, time, pH and water activity or relative humidity.

Results in terms of degree of glycation are shown in Table 9 below.

TABLE 9
Results

| | | Weight ratio of the | | | | |
|---|---|---|---|---|---|---|
| | | composition (A) | | | | |
| | | comprising SF to | | | | Degree |
| Dry | | composition (B) | | | Target | of |
| Substance | | comprising | Initial | Adjusted | residence | glycation |
| (%) | Name* | dextrose | PH | PH | time (s) | (%) |
| 10 | Trial 1 (a)** | 1:1 | 6.64 | 7.97 | 10 | 5.3 |
| | | | | | 30 | 4.7 |
| | Trial 2 (a)** | 1:5 | 6.56 | 8.06 | 10 | 6.8 |
| | | | | | 30 | 6.8 |
| | Trial 3 (a) | 1:1 | 6.56 | 8.00 | 10 | 8.0 |
| | | | | | 30 | 5.1 |
| | Trail 3 (b) | 1:1 | 6.66 | 8.03 | 10 | 8.6 |
| | | | | | 30 | 4.5 |
| | Trial 4 (a) | 1:1 | 6.56 | 8.13 | 10 | 8.0 |
| | | | | | 30 | 3.8 |

*the (a) and (b) in the trial names refer to reactants prepared according to methods (a) or (b) respectively
**in trials 1 and 2 SF sample was sieved via a 100 μm sieve prior to mixing with dextrose solution, instead of centrifugation

The proteins could be successfully glycated at a much faster rate than using the conventional methods in a dry state or wet state, which take several hours or even days, whilst still achieving high rates of glycation.

The highest rate of glycation was observed when the composition (C) had:
a dry substance of about 10 wt %,
a 1:1 weight ratio of the protein composition to the glucose composition,
a pH of 8 and
the residence time in the reactor was 10 s.

### Example 4: Glycation of sHWP with Glucose in an 8 Channel Lab-Scale Microreactor

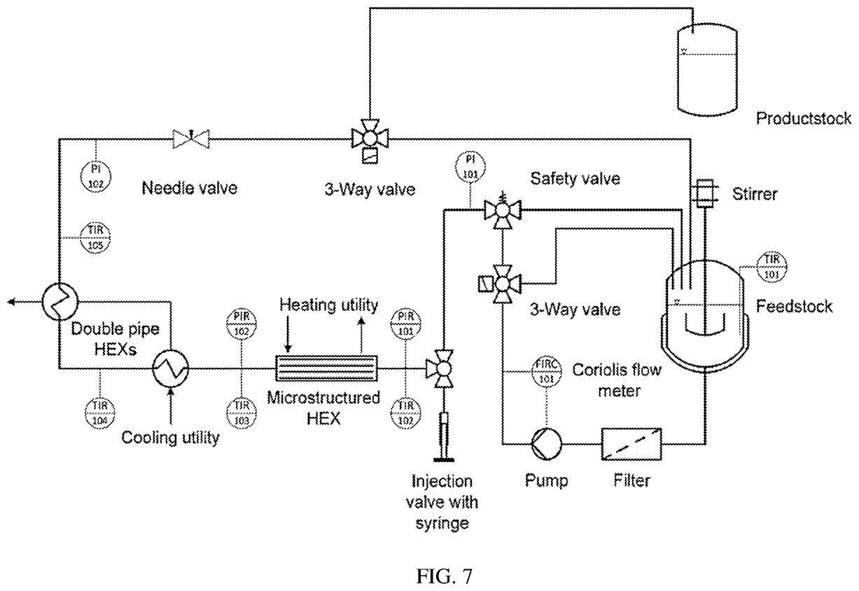

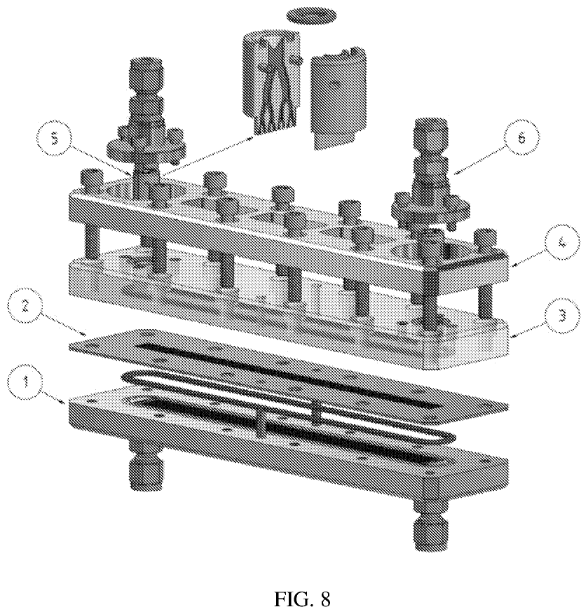

For upscaling experiments, the most promising conditions from the runs of Example 1 were used in a lab-scale 8 channel microreactor system as shown in the process flow diagram in FIG. 7. FIG. 8 illustrates the configuration of the microreactor with eight parallel microchannels in an explosion image. All microchannels share the same base geometry with a squared cross-section of 0.5×0.5 mm² and length of 185 mm. In FIG. 8 (1) is the base plate, (2) is the microstructured foil, (3) is the PMMA cover, (4) are the steel cover with cut-outs, (5) are the flow distributors and (6) are compression fittings.

FIGS. 7 and 8 are originally disclosed by Sadir et al. in the publication “Numerical and Experimental Investigation of Flow Maldistribution due to Blockage in Microstructured Heat Exchanger” in the Journal of Fluid Flow, Heat and Mass Transfer (JFFHMT), Volume 8, 2021 (publication in progress).

The same reactants as in Example 1 were used and prepared in the same way for injection into the lab-scale microreactor.

Besides the parameters in table 10 below, all experiments were carried out under the following conditions:
pH-Value: 8.0
sHWP to glucose composition ratio: 1:1
Number of passes: 6
Number of microchannels: 8
Product inlet temperature: 40° C.
Hot Utility mass flow rate: 1000 g min-1

Due to the high hot utility mass flow rate, these experiments do not show a significant drop in terms of hot utility outlet temperature. Therefore, table 10 shows the average wall temperature, which is calculated as the mean value between hot utility inlet and outlet temperature. The residence time calculation included the pipe length from the outlet of the micro-reactor towards the entry of the cooling utility assuming limited temperature loss. To reduce the potential risk of blocking the micro-channels the solids content was limited to 10 wt %.

For trials 9 and 10, the flow rate was adjusted so that each pass through the microreactor resulted in a residence time of about 10 seconds. in total the processed produced was pumped 6 times through the same micro-reactor resulting in about 55-60 seconds overall residence time. The extent of glycation was measured at 10 seconds, 30 seconds and 60 seconds.

TABLE 10

| | Solids | Product | Number | | Average | Average |
|---|---|---|---|---|---|---|
| | content | mass | of passes | Total | wall | product |
| | % | flow | in the | residence | temper- | outlet |
| Trial | (w/w) | rate | system | time | ature | temperature |
| 9 | 10 | 19 g | 6 | 55.2 s | 89.2° C. | 85.1° C. |
| | | min⁻¹ | | | | |
| 10 | 10 | 19 g | 6 | 55.2 s | 99.0° C. | 88.2° C. |
| | | min⁻¹ | | | | |

Surprisingly, for trial 9 at around 90° C. of micro-reactor wall temperature, after just one pass (about 10 seconds residence time) through the microreactor the degree of glycation was found at ca. 13%, whereas the subsequent passes to reach 30 seconds and 60 seconds residence time yielded 11 and 12% degrees of glycation. This indicated that the maximum level of glycation at 90° C. is already reached at just 10 seconds residence time for this particular system.

For trial 10, the micro-reactor wall temperature was increased to around 100° C. This trial showed a steady increase of glycation from ca. 2-3% after 10 seconds to ca. 7% after 60 seconds. However, at this reaction temperature some early signs of fouling and blockage could be observed in some of the micro-channels after 60 seconds. This could explain the overall lower degree of functionalization of the protein compared to trial 9, despite the higher temperature used in trial 10.

Finally, it can be seen that the same reaction conditions as in Example 1 can be successfully applied to a larger lab-scale reaction in an 8 channel microreactor system.

1. A process for preparing modified protein comprising the following steps:
a) Mixing a composition of protein(s) (A) and a composition of carbohydrate(s) (B) to form a composition (C) in an aqueous medium;
b) Optionally adjusting the pH of the composition (C), preferably to a pH of from 6.0 to 9.0, more preferably from 6.5 to 9.0, even more preferably from 7.0 to 8.5, most preferably from 7.0 to 8.0;
c) Adding the composition (C) into a microdevice;
d) Reacting the protein(s) with the carbohydrate(s) in the microdevice to obtain a composition comprising modified protein(s) (D).

2. The process according to claim 1 wherein the composition of protein(s) (A) is a composition comprising one or more plant-based protein(s), dairy protein(s), single cell protein(s), and fungal protein(s).

3. The process according to claim 1 wherein the composition of protein(s) (A) is a composition comprising one or more of cereal and legume protein(s), preferably one or more of wheat, corn, soybean and pea protein(s), most preferably one or more of wheat and soybean protein(s).

4. The process according to claim 1, wherein the composition of carbohydrate(s) (B) is a composition comprising, essentially consisting of or consisting of one or more of monosaccharide(s), disaccharide(s), and oligosaccharide(s).

5. The process according to claim 4 wherein the composition of carbohydrate(s) (B) comprises one or more of glucose, allulose, mannose, fructose, rhamnose, galactose, maltose, lactose, lactulose, and isomaltose, preferably the composition of carbohydrate(s) comprises, essentially consists of or consists of glucose.

6. The process according to claim 1, wherein the weight ratio of composition of protein(s) (A) to composition of carbohydrate(s) (B) in the composition (C) ranges from 1:10 to 10:1.

7. The process according to claim 1, wherein the composition (C) has a dry substance content of from 5 to 50 wt %.

8. The process according to claim 1, wherein the pH is adjusted in step (b) with a base, preferably sodium hydroxide.

9. The process according claim 1, wherein the microdevice comprises micro-heat exchangers and/or micro-reactors and optionally micro-mixer(s), and is suitable for the reaction of protein with carbohydrate.

10. The process according to claim 1, wherein the reaction in step d) takes place in the microdevice:
at a temperature of from 60 to 120° C., preferably 70 to 110° C., more preferably 80 to 100° C., most preferably 85 to 95° C. and
for a duration of less than 150 seconds, preferably less than 120 seconds, more preferably less than 110 seconds, even more preferably less than 100 seconds, most preferably from 1 to 90, 80, 70, 60, 50, 40, 30, 20 or 10 seconds.

11. The process according to claim 1, further comprising step (e) wherein the composition of modified protein(s) (D), optionally mixed with a further composition of carbohydrate(s) (B′), which can be same or different from the composition (B), is reinjected back into the same microdevice and/or into a second microdevice to increase the degree of modification of protein(s) in order to obtain a composition of modified protein(s) (D′).

12. The process according to claim 1, further comprising step (f) wherein the composition of modified protein(s) (D) or (D′) is
optionally purified and
incorporated into a food, feed, personal care, cosmetic, pharmaceutical, paper or corrugated board product comprising at least one other ingredient.

13. A wheat protein(s) glycated with mono-, di-, oligo- or polysaccharide(s).

14. A food, feed, personal care, cosmetic, pharmaceutical, paper or corrugated board product comprising the glycated wheat protein according to claim 13 and at least one other ingredient.

15. The process of claim 1, additionally comprising the step of combining the composition comprising modified protein(s) (D) with at least one other ingredient to form a food, feed, personal care, cosmetic, pharmaceutical, paper, or corrugated board product.

16. (canceled)

17. The wheat protein of claim 13, wherein the wheat protein is glycated with one or more of glucose, mannose, galactose, rhamnose, fructose, maltose, isomaltose, maltulose, mannobiose, and lactose.

18. The process of claim 1, wherein the weight ratio of composition of protein(s) (A) to composition of carbohydrate(s) (B) in the composition (C) ranges from 1:5 to 5:1.

19. The process of claim 1, wherein the weight ratio of composition of protein(s) (A) to composition of carbohydrate(s) (B) in the composition (C) ranges from 1:1 to 5:1.

20. The process of claim 1, wherein the composition (C) has a dry substance content of 10 to 40 wt %.

21. The process of claim 1, wherein the composition (C) has a dry substance content of 20 to 35 wt %.
